(19) United States
(12) Patent Application Publication
(10) Pub. No.: US 2021/0403825 A1
(43) Pub. Date: Dec. 30, 2021

(54) HYDROLYTICALLY LABILE PRO-FRAGRANCES CONTAINING ALPHA, BETA UNSATURATED ESTERS

(71) Applicant: Henkel AG & Co. KGaA, Duesseldorf (DE)

(72) Inventors: Sascha SCHAEFER, Mettmann (DE); Nora SCHMIDT, Hanau (DE); Iris DIENWIEBEL, Lengerich (DE)

(21) Appl. No.: 17/276,491

(22) PCT Filed: Sep. 11, 2019
(86) PCT No.: PCT/EP2019/074194

§ 371 Date: Mar. 16, 2021

(30) Foreign Application Priority Data
Sep. 17, 2018 (DE) 10 2018 122 761.2

(51) Int. Cl. C11B 9/00; C07C 255/31; C07C 255/41; C07C 253/30

(52) U.S. Cl. CPC C11B 9/003; C07C 255/31; C07C 255/41; C07C 2601/16; C11B 9/0034; C11B 9/0065; C07C 2601/08; C07C 253/30

(57) ABSTRACT

Pro-fragrances may have the formula (I) as disclosed herein. Compositions may include the pro-fragrances, such as a laundry product, a home care product, and an insect repellant. A method for preparing the compound of formula (I) are also disclosed. Such pro-fragrances increase the longlastingness of a fragrance, the stability of a fragrance, and the adhesion of a fragrance.

### CROSS-REFERENCE TO RELATED APPLICATIONS

The present application is a national stage entry according to 35 U.S.C. § 371 of PCT application No.: PCT/EP2019/074194 filed on Sep. 11, 2019; which claims priority to German Patent Application Serial No.: 10 2018 122 761.2 filed on Sep. 17, 2018; all of which are incorporated herein by reference in their entirety and for all purposes.

### TECHNICAL FIELD

The present disclosure pertains to the field of pro-fragrances and relates to a pro-fragrance compound of formula (I). Furthermore, the disclosure relates to compositions comprising the compound of formula (I), including a laundry detergent composition or product, a home care composition or product, and an insect repellant (composition). The disclosure further relates to a method for preparing the compound of formula (I) and uses of a compound of formula (I) for increasing the longlastingness and/or stability of a fragrance and for improving the adhesion of a fragrance to textiles.

### BACKGROUND

It is known that the scent experience of volatile fragrances and perfumes in washing products, cleaning products, insect repellants, and body care products is often limited to a short period of time due to the high volatility of the fragrance compounds. Furthermore, some fragrance compounds degrade, polymerize or structurally alter over time and thus lose their scent. These drawbacks may become more pronounced when the composition is exposed to sunlight, high temperatures, air or water.

These properties make it difficult to store products containing such fragrance compounds and perfumes over a longer period of time before use. In addition, they make the use of such fragrance compounds in liquid or gel-like compositions, such as laundry products, cleaning agents, insect repellants or body care products challenging.

To counter some of these effects, the addition of antioxidants, metal sequestering agents and other stabilizers to the fragrance-containing composition is well known. However, these additional compounds often show only insufficient stabilization.

Therefore, there is still need in the art for alternative means to stabilize fragrance compounds such as ketone- or aldehyde-based fragrances in compositions to increase the longlastingness of the desired scent experience. It is particularly desirable to avoid addition of external stabilizing compounds to reduce the number of compounds of the respective composition to control release of the fragrance compound such that it is either released at a desired point of time or over a defined period of time.

### SUMMARY

The stability and scent longlastingness of a fragrance compound in compositions can be extended by forming a pro-fragrance compound with an α,β-unsaturated ester group from a fragrance ketone or aldehyde, said pro-fragrance having the property to release the fragrance by hydrolysis. Using such pro-fragrances, the scent impression of compositions or products, such as laundry products, home care products, air care products and insect repellants can be prolonged.

In a first aspect, a pro-fragrance compound may have that of formula (I)

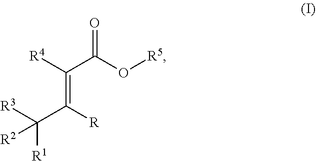

wherein

R, R¹, R², R³ and R⁵ are, independently of each other, selected from H, linear or branched, saturated or unsaturated, substituted or unsubstituted hydrocarbon groups including up to 20 carbon atoms and optionally up to 6 heteroatoms,

or

wherein R and one of the groups of R¹, R² and R³ form a substituted or unsubstituted hydrocarbon ring selected from substituted or unsubstituted cycloalkyl, cycloalkenyl or aryl groups including up to 12, preferably up to 8, more preferably 5 to 6, most preferably 5 carbon atoms, or substituted or unsubstituted heterocycloalkyl, heterocycloalkenyl or heteroaryl groups including up to 12, preferably up to 8, more preferably 5 to 6 carbon atoms, and 1 to 6, preferably 1 to 4 heteroatoms selected from O, S and N, or linear or branched, substituted or unsubstituted alkylcycloalkyl, alkenylcycloalkyl, alkylcycloalkenyl, alkenylcycloalkenyl, alkylaryl or alkenylaryl groups including up to 20, preferably up to 12 carbon atoms, or linear or branched, substituted or unsubstituted heteroalkylcycloalkyl, heteroalkenylcycloalkyl, heteroalkylcycloalkenyl, heteroalkenylcycloalkenyl, heteroalkylaryl or heteroalkenylaryl groups including up to 20, preferably up to 12 carbon atoms, and 1 to 6, preferably 1 to 4 heteroatoms selected from O, S and N, or linear or branched, substituted or unsubstituted heteroalkylheterocycloalkyl, heteroalkenylheterocycloalkyl, heteroalkylheterocycloalkenyl, heteroalkenylheterocycloalkenyl, heteroalkylheteroaryl or heteroalkenylheteroaryl groups including up to 20, preferably up to 12 carbon atoms, and 1 to 6, preferably 1 to 4 heteroatoms selected from O, S and N, and wherein the remaining groups are as defined above, and wherein R⁵ is as defined above;

wherein R⁴ is an electron-withdrawing group, preferably an ester group, a thioester group, a semicarbamide group, a hydrazone group, an amide group, a nitro group, a ketone group, an aldehyde group, a halogen atom, —CF₃, —C—N, or a carboxylic acid group, more preferably R⁴ is an ester group, —CF₃, —F, —Cl, or —C—N, most preferably R⁴ is —C—N;

with the proviso that at least one of R, R¹, R², and R³ is not hydrogen and that R, R¹, R², and R³ together with the carbon atoms to which they are attached are derived from a fragrance ketone or aldehyde of the formula (II)

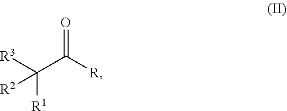

wherein R, R¹, R², and R³ are as defined above.

Said fragrance ketone or aldehyde can be released as the compound of formula (II) from the pro-fragrance compound of the formula (I).

In a second aspect, a composition may include a pro-fragrance compound of formula (I).

In a third, fourth and fifth aspect, the composition relates to a laundry product, a home care product and an insect repellant, each comprising a pro-fragrance compound of formula (I).

In a sixth aspect, a method for preparing a pro-fragrance compound of formula (I) may include:

(i) reacting a compound of formula (XIII)

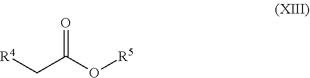

with a fragrance compound of formula (II), as defined above,

wherein R⁴ is an electron-withdrawing group, as defined above;

and wherein R⁵ is selected from H, or a linear or branched, saturated or unsaturated, substituted or unsubstituted hydrocarbon groups including up to 20 carbon atoms and optionally up to 6 heteroatoms.

In addition, a pro-fragrance compound of formula (I) may increase the longlastingness of a fragrance in compositions, in comparison to a corresponding free fragrance compound according to formula (II).

Furthermore, a pro-fragrance compound may increase the stability of a fragrance in compositions, in comparison to a corresponding free fragrance compound according to formula (II).

Finally, a pro-fragrance compound may increase the adhesion of a fragrance to textiles, in comparison to a corresponding free fragrance compound according to formula (II).

Preferably, the pro-fragrance compound of formula (I) is suitable to release a fragrance compound over time, more preferably by hydrolysis, more preferably the hydrolysis occurs by contact of the pro-fragrance compound of formula (I) with added water. Most preferably, the hydrolysis occurs by contact of the pro-fragrance compound of formula (I) with moisture from the air and sweat. Preferably, the hydrolysis of the pro-fragrance compound releases the fragrance compound, which provides the desired scent experience. The released fragrance compound is a compound of formula (II).

### DETAILED DESCRIPTION

“One or more”, as used herein, relates to “at least one” and comprises 1, 2, 3, 4, 5, 6, 7, 8, 9 or more of the referenced species. Similarly, “at least one” means “one or more”, i.e. 1, 2, 3, 4, 5, 6, 7, 8, 9 or more. “At least one”, as used herein in relation to any component, refers to the number of chemically different atoms or molecules, i.e. to the number of different types of the referenced species, but not to the total number of atoms or molecules. For example, “at least one further component” means that at least one type of component falling within the definition can be part of the composition, but that also two or more different component types falling within this definition can be present.

“Up to x carbon atoms”, for example “up to 20 carbon atoms”, refers to (hydrocarbon) groups with a minimum of 1 and a maximum of 20 carbon atoms, the numbers being integers. The possibility of 0 carbon atoms is excluded, when using the wording of “up to x carbon atoms”. In case of unsaturated, branched or cyclic hydrocarbon groups the lower limit is adapted accordingly, i.e. to 2 or 3 carbon atoms.

The optional amount of “up to x heteroatoms”, for example “up to 6 heteroatoms” relates to groups which may each contain 0 to 6 heteroatoms. Since this feature is optional, the possibility of 0 heteroatoms is included in the scope of said term, if not explicitly stated otherwise, for example be reference to a heteroalkyl group, in which case the presence of at least one heteroatom is mandatory.

In a preferred embodiment, the wording “up to x hours” is related to the increase of longlastingness of a fragrance in compositions, when using the pro-fragrance compound, in comparison to a corresponding free fragrance compound.

Numeric values specified without decimal places refer to the full value specified with one decimal place. For example, “99%” means “99.0%” if not stated otherwise.

The expressions “approx.” or “about”, in conjunction with a numerical value, refer to a variance of ±10% relative to the given numerical value, preferably ±5%, more preferably ±1%, if not explicitly stated otherwise.

All percentages given herein in relation to the compositions or agents relate to weight % (wt.-%) relative to the total weight of the respective composition or agent, if not explicitly stated otherwise.

The terms “product” and “agent” and “composition”, such as laundry product or laundry agent or laundry composition, are interchangeably used in this application.

Phrases, such as “increased longlastingness of a fragrance compound” or “improved stability of a fragrance compound” or “improved adhesion to textiles” refer to a direct comparison between the fragrance compound bound in the pro-fragrance compound and the corresponding free fragrance compound according to formula (II) without any pro-group, if not explicitly stated otherwise. The effect of using a pro-fragrance compound in a composition, in comparison to a free fragrance compound, is demonstrated in Tables 1 and 2.

These and other aspects, features and advantages become apparent to the skilled person in the following detailed description and claims. Each feature can be used in any other aspect. Furthermore, the examples contained herein are intended to describe and illustrate the embodiments, but do not restrict them and in particular, the embodiments are not limited to these examples. While the formulae herein, in particular Formulae (I), (III), (IV) and the specific compounds disclosed herein, show only one of the cis/trans isomers, the other isomer is also included, as typically both isomers (cis/trans) are obtained during synthesis. “cis/trans” isomers relates to the orientation of the groups on the two carbon atoms that form the C═C double bond.

“Corresponding free fragrance compound”, as used herein, relates to a fragrance compound structurally identical to that released from the pro-fragrance.

When one or more groups of R, R¹, R² R³ or R⁵ are substituted, the substituent can be chosen from every suitable substituent that is known to the person skilled. Preferably, each of the groups of R, R¹, R², R³ and/or R⁵ may contain any one or more, for example 1 to 10 of each or different, of the following substituents: halogen, ester, ether, hydroxyl, amine, amide, ketone, aldehyde, carboxyl, linear C1-C20 alkyl, branched C3-C20 alkyl, linear C2-C20 alkenyl, branched C3-C20 alkenyl, linear C2-C20 alkynyl, branched C4-C20 alkynyl; C3-C20 cycloalkyl, C3-C20 cycloalkenyl, or aryl; linear C4-C20 alkylcycloalkyl, C4-C20 alkylcycloalkenyl, C4-C20 alkenylcycloalkyl, C4-C20 alkenylcycloalkenyl, C5-C20 alkynylcycloalkyl or C5-C20 alkynylcycloalkenyl, or branched C5-C20 alkylcycloalkyl, C5-C20 alkylcycloalkenyl, C5-C20 alkenylcycloalkyl, C5-C20 alkenylcycloalkenyl, C6-C20 alkynylcycloalkyl or C6-C20 alkynylcycloalkenyl; or linear C7-C20 alkylaryl, C8-C20 alkenylaryl or C8-C20 alkynylaryl, or branched C8-C20 alkylaryl, C8-C20 alkenylaryl or C9-C20 alkynylaryl or combinations thereof. The substituents may be further substituted by one or more of the groups above and each substituent may contain one or more heteroatom(s), such as, O, N, S, and P. The substituents may replace one or more of —H, —C—, —CH—, —CH₂— and/or —CH₃ in the groups R, R¹, R² R³ or R⁵. Preferred substituents are C1 to C6 alkyl, ketone, alkylketone, ester, alkylester methoxy and methoxy groups, more preferably methyl, ethyl or alkylester groups. In various embodiments, the substituents are not themselves substituted. It is further understood that the substituted group is not substituted with the same group. i.e. an alkyl group is not further substituted with an alkyl group. In this context, the following terms have the following preferred meanings: “Ester group”, as used herein, relates to a group of formula —C(═O)—O—R′ or R′—C(═O)—O—. “Ether group”, as used herein, relates to a group of formula R′—O—. “Amide”, as used herein, relates to a group —(C═O)—NR′—R′ or R′—(C═O)—NR′—. “Amine”, as used herein, relates to a group —N(R′)₂. “Ketone” and “aldehyde” mean a group of formula —(C═O)— or —(C═O)—H. “Carboxyl” means —COOH. In all the afore-mentioned formulae, R′ can be H or up to C20, preferably up to C10, alkyl, alkenyl, alkynyl, aryl or the respective hetero variants thereof. These definitions also apply to the corresponding definition of R⁴.

The maximum number of carbon atoms does not include carbon atoms of optional present substituents of the groups of R, R¹, R², R³ or R⁵. That means that when R contains up to 20 carbon atoms, the substituent of R may contain further carbon atoms in the substituents so that more than 20 carbon atoms, for example 22 carbon atoms, are included in R in total.

The compound is a pro-fragrance. The term “pro-fragrance” describes in general derivatives of alcohol, ester, ether, aldehyde and ketone fragrances, which release the original alcohol, ester, ether, aldehyde and ketone under suitable conditions. The pro-fragrance consists of a ketone or aldehyde derivative, which is bound to an ester-containing anchor or protection group.

The ester-containing anchor or protection group is used to avoid or slow down the alteration, degradation, polymerization or evaporation of the aldehyde or ketone fragrance compound over time. The aldehyde or ketone fragrance is released from the pro-fragrance compound under suitable conditions, preferably by hydrolysis. The release may occur during a washing process or during wearing of clothes laundered with a composition comprising the pro-fragrances.

The exemplary release of a ketone fragrance compound from the pro-fragrance compound is demonstrated in the following scheme:

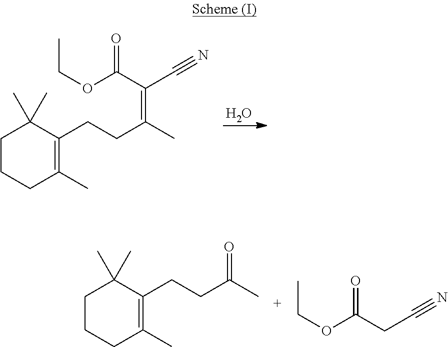

Preferably, the ester-containing anchor or protection group at the aldehyde or ketone fragrance compound enables a prolonged stability or slower evaporation rate of the fragrance compound and an extended fragrance effect in compositions or agents comprising the pro-fragrance, compared to compositions, comprising unprotected aldehyde or ketone fragrances (Tables 1 and 2).

The fragrance bound in the pro-fragrance compound is derived from a ketone or aldehyde fragrance compound, preferably from a ketone fragrance compound.

In a preferred embodiment, the pro-fragrance compound is suitable to release a ketone fragrance compound selected from methyl-beta-naphthyl ketone, muskindanone (1,2,3,5,6,7-hexahydro-1,1,2,3,3-pentamethyl-4H-inden-4-one), tonalid (6-acetyl-1,1,2,4,4,7-hexamethyltetralin), alpha-damascone, beta-damascone, delta-damascone, iso-damascone, damascenone, hedione (methyl dihydrojasmonate), menthone (2-isopropyl-5-methylcyclohexanone), carvone (methyl-5-(prop-1-en-2-yl)cyclohex-2-enone), camphor, koavon (3,4,5,6,6-pentamethylhept-3-en-2-one), fenchone, alpha-ionone, beta-ionone, gamma-methyl-ionone, dihydro-beta-ionone, fleuramone (2-heptylcyclopentanone), frambinone (4-(4-hydroxy-phenyl-butan-2-one), frambinone methyl ether, dihydrojasmone, cis-jasmone, 1-(1,2,3,4,5,6,7,8-octahydro-2,3,8,8-tetramethyl-2-naphthalenyl)-ethan-1-one and isomers thereof, methylcedrenylketone, acetophenone, methylacetophenone, para-methoxyacetophenone, methyl-beta-naphtylketone, benzyl acetone, benzophenone, para-hydroxyphenylbutanone, celery-ketone (3-methyl-5-propylcyclohex-2-en-1-one), 6-isopropyldecahydro-2-naphtone, dimethyloctenone, frescomenthe (2-butane-2-yl-cyclohexane-1-one), 4-(1-ethoxyvinyl)-3,3,5,5-tetramethylcyclohexanone, methylheptenone, 2-(2-(4-methyl-3-cyclohexene-1-yl)propyl)cyclopentanone, 1-(p-menthene-6(2)yl)-1-propanone, 4-(4-hydroxy-3-methoxyphenyl)-2-butanone, nectaryl (2-(2-(4-methyl-3-cyclohexen-1-yl)propyl)cyclopentanone), 2-acetyl-3,3-dimethylnorbornane, 6,7-dihydro-1,1,2,3,3-pentamethyl-4-(5H)-indanone, 4-damascol, dulcinyl (4-(1,3-benzodioxol-5-yl)butane-2-one), hexalone (1-(2,6,6-trimethyl-2-cyclohexene-1-yl)-1,6-heptadiene-3-one), isocyclemone E 1-(2,3,8,8-tetramethyl-1,2,3,4,5,6,7,8-octahydro-2-naphthalenyl)ethanone, methylnonylketone, methylcyclocitrone, methyllavendelketone, orivone (4-tert-amylcyclohexanone), 4-tert-butyl cyclohexanone, delphone (2-pentyl cyclopentanone), muscone (CAS 541-91-3), neobutenone (1-(5,5-dimethyl-1-cyclo-hexenyl)pent-4-en-1-one), plicatone (CAS 41724-19-0), veloutone (2,2,5-trimethyl-5-pentylcyclopentane-1-one), 2,4,4,7-tetramethyl-oct-6-en-3-one, and tetrameran (6,10-dimethylundecene-2-one), more preferably from dihydro-beta-ionone, hedione, nectaryl, frambinone, frambinone methyl ether, fleuramone, and benzyl acetone. Preferably, the released ketone fragrance compound is hedione, dihydro-beta-ionone, fleuramone, frambinone, frambinone methyl ether, nectaryl or benzyl acetone.

In another embodiment, the fragrance bound in the pro-fragrance compound is derived from suitable aldehyde fragrances: adoxal (2,6,10-trimethyl-9-undecenal), anisaldehyde (4-methoxybenzaldehyde), cymale (3-(4-isopropylphenyl)-2-methylpropanal), ethylvanillin, florhydral (3-(3-isopropylphenyl)butanal), helional (3-(3,4-methylendioxyphenyl)-2-methylpropanal), heliotropin, hydroxycitronellal, lauraldehyde, lyral (3-und 4-(4-hydroxy-4-methylpentyl)-3-cyclohexen-1-carboxaldehyde), methylnonylacetaldehyde, lilial (3-(4-tert-butylphenyl)-2-methylpropanal), phenylacetaldehyde, undecylenaldehyde, vanillin, 2,6,10-trimethyl-9-undecenal, 3-dodecen-1-al, alpha-n-amyl cinnamic aldehyde, melonal (2,6-dimethyl-5-heptenal), 2,4-dimethyl-3-cyclohexen-1-carboxaldehyde (triplal), 4-methoxybenzaldehyde, benzaldehyde, 3-(4-tert-butylphenyl)-propanal, 2-methyl-3-(para-methoxyphenyl)propanal, 2-methyl-4-(2,6,6-timethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzylaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal, 2,6-dimethyl-5-heptenal, 4-(tricyclo[5.2.1.0(2,6)]-decyliden-8)-butanal, octahydro-4,7-methane-1H-indencarboxaldehyde, 3-ethoxy-4-hydroxybenzaldehyde, para-ethyl-alpha,alpha-dimethylhydrocinnamaldehyde, alpha-methyl-3,4-(methylendioxy)-hydrocinnamaldehyde, 3,4-methylendioxybenzaldehyde, alpha-n-hexylcinnamaldehyde, m-cymene-7-carboxaldehyde, alpha-methylphenylacetaldehyde, 7-hydroxy-3,7-dimethyloctanal, undecenal, 2,4,6-trimethyl-3-cyclohexen-1-carboxaldehyde, 4-(3)(4-methyl-3-pentenyl)-3-cyclohexencarboxaldehyde, 1-dodecanal, 2,4-dimethylcyclohexen-3-carboxaldehyde, 4-(4-hydroxy-4-methylpentyl)-3-cylohexen-1-carboxaldehyde, 7-methoxy-3,7-dimethyloctan-1-al, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexen-1-carboxaldehyde, 5- or 6-methoxyhexahydro-4,7-methanindan-1- or -2-carboxaldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexencarboxaldehyde, 7-hydroxy-3,7-dimethyl-octanal, trans-4-decenal, 2,6-nonadienal, para-tolylacetaldehyde, 4-methylphenylacetaldehyde, 2-methyl-4-(2,6,6-trimethyl-1-cyclohexen-1-yl)-2-butenal, ortho-methoxycinnamaldehyde, 3,5,6-trimethyl-3-cyclohexencarboxaldehyde, 3,7-dimethyl-2-methylen-6-octenal, phenoxyacetaldehyde, 5,9-dimethyl-4,8-decadienal, peonies aldehyde (6,10-dimethyl-3-oxa-5,9-undecadien-1-al), hexahydro-4,7-methanindan-1-carboxaldehyde, 2-methyloctanal, alpha-methyl-4-(1-methylethyl)benzolacetaldehyde, 6,6-dimethyl-2-norpinen-2-propionaldehyde, para-methylphenoxyacetaldehyde, 2-methyl-3-phenyl-2-propen-1-al, 3,5,5-trimethylhexanal, hexahydro-8,8-dimethyl-2-naphthaldehyde, 3-propylbicyclo[2.2.1]-hept-5-en-2-carbaldehyde, 9-decenal, 3-methyl-5-phenyl-1-pentanal, methylnonylacetaldehyde, hexanal, trans-2-hexenal and mixtures thereof.

In preferred embodiments, the composition comprises at least one further component selected from one or more of: anionic surfactants, cationic surfactants, amphoteric surfactants, nonionic surfactants, acidifiers, alkalizing agents, anti-crease compounds, antibacterial/antimicrobial substances, antioxidants, antideposition agents, antistatic agents, bitter substances, bleaching agents, bleach activators, bleach stabilizers, bleach catalysts, builder substances, corrosion inhibitors, ironing aids, cobuilders, further fragrances or pro-fragrances, shrinkage inhibitors, electrolytes, emulsifiers, enzymes, enzyme stabilizers, protease inhibitors, colorants, dyes, dye transfer inhibitors, fluorescent agents, fungicides, germicides, metal sequestering agents, odor-complexing substances, auxiliary agents, hydrotropics, rinse aids, complexing agents, preservatives, optical brighteners, perfume carriers, pearl-luster agents, pH control agents, phobing and impregnating agents, polymers, swelling and sliding fasteners, foam inhibitors, layered silicates, dirt-repellent substances, organic solvents, silicone oils, soil-release active substances, UV-protective substances, viscosity regulators, thickeners, discoloration inhibitors, greying inhibitors, structurants, vitamins, fabric softeners and/or water.

Preferably, the compositions further contain anionic surfactants, cationic surfactants, amphoteric surfactants or nonionic surfactants, builder substances, cobuilders, further fragrances or pro-fragrances, emulsifiers enzymes, enzyme stabilizers, organic solvents, greying inhibitors, chelating agents, crystal growth inhibitors, foam inhibitors, structurants, optical brighteners, thickeners bleaching agents, bleaching activators, pH control agents and water.

The pro-fragrance compound may be used as the only fragrance substance in a composition or an agent, but it is also possible to use mixtures of fragrances. Preferably, fragrance mixtures containing 0.01 to 95 wt.-%, more preferably 0.1 to 80 wt.-%, most preferably 0.15 to 50 wt.-% of the compound of formula (I), based on the total weight of the fragrance mixture. By the use of additional perfume compounds in the compositions, e.g., in laundry detergents or cleaning compositions, it is possible to create a variety of characteristics of the final product, which are only possible by using them in combination with the compound. For example, it is possible to divide the total perfume content (fragrance content) of a composition, for example a detergent or cleaning composition, into two portions, x and y, wherein portion x comprises the compound and portion y comprises further fragrance compounds. These further fragrance compounds may be unprotected, i.e. free fragrances and may comprise the same fragrance compounds as those incorporated in the pro-fragrances.

The fragrance compounds (or perfume compounds, with these two terms being used interchangeably herein) that may be additionally incorporated are not subject to any restrictions. Individual perfume substance compounds of natural or synthetic origin, e.g., of the type of esters, ethers, aldehydes, ketones, alcohols and hydrocarbons may thus be used as the perfume substance including perfume oils.

Suitable ketone and aldehyde compounds are already mentioned above. Fragrance compounds of the ester type include, for example, benzyl acetate, phenoxyethyl isobutyrate, p-tert-butylcyclohexyl acetate, linalyl acetate, dimethylbenzylcarbinyl acetate (DMBCA), phenylethyl acetate, ethylmethyl phenyl glycinate, allylcyclohexyl propionate, styrallyl propionate, benzyl salicylate, cyclohexylsalicylate, floramat, melusat and jasmacyclate. The ethers include, for example, benzylethylether and ambroxan; the alcohols include, for example, anethole, citronellol, eugenol, geraniol, linalool, phenylethyl alcohol and terpineol; the hydrocarbons include mainly terpenes such as limonene and pinene. However, mixtures of various fragrance substances, which jointly produce an attractive scent note are preferred.

Such fragrance compounds may also contain mixtures of natural perfume substances such as those accessible from plant sources, e.g., pine oil, citrus oil, jasmine oil, patchouli oil, rose oil or ylang-ylang oil. Also suitable are muscatel sage oil, chamomile oil, clove oil, lemon balm oil, mint oil, cinnamon leaf oil, linden blossom oil, juniper berry oil, vetiver oil, olibanum oil, galbanum oil and labdanum oil as well as orange blossom oil, neroli oil, orange peel oil and sandalwood oil.

Other traditional fragrance substances that may be used include, for example, the essential oils such as angelica root oil, anise oil, arnica blossom oil, sweet basil oil, bay oil, champaca blossom oil, silver fir oil, fir cone oil, elemi oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, cajeput oil, calamus oil, chamomile oil, camphor oil, canaga oil, cardamom oil, cassia oil, pine needle oil, copaiba balsam oil, coriander oil, spearmint oil, caraway oil, cumin oil, lavender oil, lemon grass oil, lime oil, mandarin oil, lemon balm oil, ambrette seed oil, myrrh oil, clove oil, neroli oil, niaouli oil, olibanum oil, origanum oil, palmarosa oil, patchouli oil, Peru balsam oil, petitgrain oil, pepper oil, peppermint oil, allspice oil, pine oil, rose oil, rosemary oil, sandalwood oil, celery seed oil, spike lavender oil, star anise oil, turpentine oil, thuja oil, thyme oil, verbena oil, vetiver oil, juniper berry oil, vermouth oil, wintergreen oil, ylang-ylang oil, ysop oil, cinnamon oil, cinnamon leaf oil, citronella oil, lemon oil and cypress oil as well as compounds selected from the group of ambrettolide, ambroxan, α-amylcinnamaldehyde, anethole, anise aldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerate, borneol, bornyl acetate, boisambrene forte, α-bromostyrene, damascone, damascenone, n-decylaldehyde, n-dodecylaldehyde, eugenol, eugenol methyl ether, eucalyptol, farnesol, fenchone, fenchyl acetate, geranyl acetate, geranyl formate, heliotropin, heptin carboxylic acid methyl ester, heptaldehyde, hydroquinone dimethyl ester, hydroxycinnamyl aldehyde, hydroxycinnamyl alcohol, indole, iron, isoeugenol, isoeugenol methyl ether, isosafrol, jasmine, camphor, carvacrol, carbon, p-cresol methyl ether, coumarin, p-methoxyacetophenone, methyl-n-amyl ketone, methyl anthranilic acid methyl ester, p-methylacetophenone, methyl chavicol, p-methylquinoline, methyl β-naphthyl ketone, methyl n-nonylaldehyde, nonyl alcohol, n-octylaldehyde, p-oxyacetphenone, pentadecanolide, β-phenylethyl alcohol, phenylacetaldehyde-dimethylacetal, phenylacetic acid, pulegon, safrole, salicylic acid isoamyl ester, salicylic acid methyl ester, salicylic acid hexyl ester, salicylic acid cyclohexyl ester, santalol, sandelice, skatol, terpineol, thyme, thymol, troenan, γ-undelactone, vanillin, veratrum aldehyde, cinnamyl aldehyde, cinnamyl alcohol, cinnamic acid, cinnamic acid ethyl ester, cinnamic acid benzyl ester, diphenyl oxide, limonene, linalool, linayl acetate and linalyl propionate, melusat, menthol, menthone, methyl-n-heptenone, pinene, phenyl acetaldehyde, terpinyl acetate, citral, citronellal and mixtures thereof.

In various embodiments, the compositions comprise the same fragrance compound in form of a pro-fragrance and in free form.

All fragrance substances disclosed herein, can be used in the compositions or agents in free or encapsulated form or both. Preferably, the pro-fragrance compound can be used in free or encapsulated form or both. Preferably, the pro-fragrance compound is used in non-encapsulated form. In various embodiments, the pro-fragrance compound can be combined with encapsulated fragrances. As capsules, preferably microcapsules can be used. Preferably, microcapsules, which are known in the art can be used without limitation.

In a preferred embodiment, the composition can be a liquid at 25° C., preferably a gel.

In another preferred embodiment, the composition is a solid at 25° C.

Preferably, the composition or a liquid laundry agent, home care agent or insect repellant, comprising the pro-fragrance compound has a viscosity of 50 to 100,000 mPa s at 20° C., more preferably of 1,000 to 50,000 mPa s at 20° C., preferably measured with a Brookfield viscometer.

“Liquid compositions” as used herein, relate to compositions, which are fluid or flowable at standard conditions (20° C., 1013 mbar). Liquid compositions also comprise gel-like and paste-like compositions. In particular, not-Newtonian liquids, which have a yield strength, are comprised, as well.

The water content of liquid compositions, as used herein, may be measured by Karl Fischer titration (Angewandte Chemie 1935, 48, 394-396; ISBN 3-540-12846-8 Eugen Scholz).

In a preferred embodiment, the composition, laundry product, home care product or insect repellant has a water content of 2 to 95 wt.-%, preferably 10 to 80 wt.-%, and more preferably 25 to 70 wt.-%, based on the total weight of the composition, laundry product, home care product or insect repellant. In the case of a gel, the water content can also be lower and can be 30 wt.-% or less, preferably 20 wt.-% or less, particularly 15 wt.-% or less.

The solvent system in the present compositions can be a solvent system containing water alone or mixtures of organic solvents either without or preferably with water. Preferred organic solvents include 1,2-propanediol, methanol, ethanol, 2-propanol, tert.-butanol, glycerol, dipropylene glycol, methyl propane diol and mixtures thereof. C1-C4 alkanolamines, such as monoethanolamine and triethanolamine, can also be used. Solvent systems can be absent, for example from anhydrous solid embodiments, but more typically are present at levels in the range of 0.1 wt.-% to 98 wt.-%, preferably of at least 1 wt.-% to 50 wt.-%, more preferably of 2 wt.-% to 25 wt.-%.

Suitable non-ionic surfactants are in particular, but without being limited to, ethoxylation and/or propoxylation products of alkyl glycosides and/or linear or branched alcohols each with 8 to 18 C atoms in the alkyl moiety and 3 to 20, preferably 4 to 10 alkyl ether groups. Furthermore, corresponding ethoxylation and/or propoxylation products of N-alkylamines, vicinal diols, fatty acid esters and fatty acid amides, which correspond to the above-mentioned long-chain alcohol derivatives with regard to the alkyl moiety, and of alkylphenols with 5 to 12 C atoms in the alkyl radical are useful.

Preferably, non-ionic surfactants, such as fatty alcohol polyglycol ethers, alkyl polyglucoside, or fatty acid glucamide, are used, in particular in amounts of 0.1-25 wt.-%, based on the total weight of the composition or the agent.

Suitable anionic surfactants, without being limited to, are preferably soaps and soaps containing sulphate or sulphonate groups with preferably alkali ions as cations. Suitable soaps are preferably the alkali salts of saturated or unsaturated fatty acids with 8 to 18 carbon atoms. These fatty acids can also be used in incompletely neutralized form. The useful sulfate-type surfactants include the salts of the sulfuric acid half-esters of fatty alcohols with 8 to 18 carbon atoms and the sulfation products of said non-ionic surfactants with a low degree of ethoxylation. The useful surfactants of the sulfonate-type include, for example, linear alkylbenzene sulfonates with 8 to 14 carbon atoms in the alkyl moiety, alkane sulfonates with 8 to 18 carbon atoms, and olefin sulfonates with 8 to 18 C atoms, which are formed during the reaction of corresponding mono olefins with sulfur trioxide, as well as alpha-sulfo fatty acid esters, which are formed during the sulfonation of fatty acid methyl or ethyl esters. In a preferred embodiment alkylbenzene sulfonate or alkylsulfate is present in the composition or agent, preferably in amounts of 0.1 to 40 wt.-%, based on the total weight of the composition or the agent.

Cationic surfactants are preferably selected from, but without being limited to, esterquats and/or quaternary ammonium compounds (QAV) according to the general formula (R^{I})(R^{II})(R^{III})(R^{IV})N⁺X⁻ in which R^{I} to R^{IV} represent identical or different C1 to C22 alkyl radicals, C7 to C28 arylalkyl radicals or heterocyclic radicals, wherein two or, in the case of an aromatic integration, such as in pyridine, even three radicals together with the nitrogen atom form the heterocycle, for example a pyridinium or imidazolinium compound, and X⁻ represents halide ions, sulfate ions, hydroxide ions or similar anions. QAV can be produced by reaction of tertiary amines with alkylating agents, e.g. methyl chloride, benzyl chloride, dimethyl sulfate, dodecyl bromide, but also ethylene oxide. The alkylation of tertiary amines with a long alkyl radical and two methyl groups is particularly simple. Quaternization of tertiary amines with two long radicals and one methyl group can also be carried out under mild conditions with the aid of methyl chloride. Amines, which have three long alkyl radicals or hydroxy-substituted alkyl radicals are little reactive and are quaternized e.g. with dimethyl sulfate. QAVs, which can be used, include benzalkonium chloride (N-alkyl-N,N-dimethyl-benzylammonium chloride), benzalkone B (m,p-dichlorobenzyldimethyl-C₁₂-alkylammonium chloride, benzoxonium chloride (benzyl-dodecyl-bis-(2-hydroxyethyl)-ammonium chloride), cetrimonium bromide (N-hexadecyl-N,N-trimethyl-ammonium bromide), Benzetonium chloride (N,N dimethyl-N [2-[2-[p-(1,1,3,3-tetramethylbutyl)phenoxy]-ethoxy]-ethyl]-benzylammonium chloride), dialkyldimethylammonium chlorides such as di-n-decyl-dimethylammonium chloride, didecyldimethylammonium bromide, dioctyl-dimethylammonium chloride, 1-cetylpyridinium chloride and thiazoline iodide and mixtures thereof. Preferred QAVs are benzalkonium chlorides with C8 to C22 alkyl radicals, in particular C12 to C14 alkylbenzyl-dimethylammonium chloride.

Preferred esterquats are methyl N-(2-hydroxyethyl)-N,N-di(talgacyl-oxyethyl)ammonium-metho-sulfate, bis-(palmitoyl)-ethyl-hydroxyethyl-methyl-ammonium-methosulfate ormethyl-N,N-bis(acyl-oxyethyl)-N-(2-hydroxyethyl)ammonium-methosulfate. Commercially available examples are the methylhydroxyalkyldialkoyloxyalkylammoniummethosulfates sold by Stepantex under the trademark Stepantex or the products of BASF SE known under the trade name Dehyquart or the products of Evonik known under the name Rewoquat. Preferably, esterquats are used in amounts of 0.1 to 30 wt.-%, based on the total weight of the composition or the agent.

Suitable amphoteric/zwitterionic surfactants include amine oxides and betaines.

Suitable emulsifiers can be fatty amine ethoxylates, preferably in amounts of 0.01 to 4 wt.-%, based on the total weight of the composition or the agent.

The composition or agents may further comprise water-soluble and water-insoluble, organic and inorganic builder substances. Water-soluble organic builder substances include for example, but without being limited to, polycarboxylic acids, in particular citric acid and sugar acids, monomeric and polymeric aminopolycarboxylic acids, in particular methylglycinediacetic acid, nitrilotriacetic acid and ethylenediaminetetraacetic acid as well as polyaspartic acid, polyphosphonic acids, in particular aminotris(methylenephosphonic acid), ethylenediaminetetrakis(methylenephosphonic acid) and 1-hydroxyethane-1,1-diphosphonic acid, polymeric hydroxy compounds such as dextrin, polymeric (poly)carboxylic acids, polymeric acrylic acids, methacrylic acids, maleic acids and copolymers thereof, which may also contain small amounts of polymerizable substances without carboxylic acid functionality when polymerized. Suitable, but less preferred compounds of this class are copolymers of acrylic acid or methacrylic acid with vinyl ethers such as vinyl methyl ethers, vinyl esters, ethylene, propylene and styrene, in which the proportion of acid is at least 50 wt.-%. The organic builder substances can be used, preferably for the production of liquid agents, in the form of aqueous solutions, preferably in the form of 30 to 50 wt.-% aqueous solutions. All acids mentioned are usually used in the form of their water-soluble salts, in particular their alkali salts.

Organic builder substances can be comprised in compositions or agents, in amounts of up to 40 wt.-%, preferably up to 25 wt.-% and more preferably from 0.1 wt.-% to 8 wt.-%. Amounts near this upper limit are preferably used in pasty or liquid, in particular water-containing agents. Laundry after-treatment agents, such as fabric softeners, may also be free of organic builder. It may be preferred that the composition comprises low levels of phosphate salt and/or zeolite, for example from 0.1 to 10 wt.-%, preferably from 1 to 5 wt.-%. Optionally, the composition may be free of strong builder.

Alkali silicates and aluminum silicates and polyphosphates, preferably sodium triphosphate, are particularly suitable as water-soluble inorganic builder materials. As water-insoluble, water-dispersible inorganic builder materials, crystalline or amorphous alkali metal aluminum silicates, if desired, can be used in quantities of up to 50 wt.-%, preferably not more than 40 wt.-%, and in liquid media, in particular from 1 wt.-% to 5 wt.-%. Among these, crystalline sodium aluminum silicates in detergent quality, in particular zeolites A, P and, where appropriate, X, are preferred. Quantities near this upper limit are preferably used in solid, particulate agents. In particular, suitable aluminum silicates have no particles with a grain size of more than 30 μm and preferably consist of at least 80 wt.-% of particles with a size of less than 10 μm.

In preferred embodiments, the builder materials are selected from zeolites, polycarboxylates or sodium citrate.

In further preferred embodiments, builder substances, if desired, can be present in compositions or agents in amounts of up to 60 wt.-%, preferably of 1 wt.-% to 40 wt.-%. Laundry after treatment agents, such as softeners, are preferably free of inorganic builder substances.

The bleach activators used can be compounds, which under perhydrolysis conditions give aliphatic peroxocarboxylic acids with preferably 1 to 10 carbon atoms, in particular 2 to 4 carbon atoms, and/or optionally substituted perbenzoic acid. Suitable substances are substances, which include O and/or N acyl groups of the carbon atom number mentioned-above and/or optionally substituted benzoyl groups. Multiple acylated alkylene diamines, preferably tetraacetylethylenediamine (TAED), acylated triazine derivatives, preferably 1,5-diacetyl-2,4-dioxo-hexahydro-1,3,5-triazine (DADHT), acylated glycoluriles, in particular tetraacetylglycoluril (TAGU), N-acylimides, in particular N-nonanoylsuccinimide (NOSI), acylated phenolsulfonates, in particular n-nonanoyl or isononanoyloxybenzenesulfonate (n- or iso-NOBS), carboxylic anhydrides, in particular phthalic anhydride, acylated polyhydric alcohols, in particular triacetin, ethylene glycol diacetate, 2,5-diacetoxy-2,5-dihydrofuran and enol ester, and acetylated sorbitol and mannitol or mixtures thereof (SORMAN), acylated sugar derivatives, in particular pentaacetyl glucose (PAG), pentaacetyl fructose, tetraacetyl xylose and octaacetyllactose as well as acetylated, optionally N-alkylated glucamine and gluconolactone, and/or N-acylated lactams, for example N-benzoylcaprolactam, are preferred. Hydrophilic substituted acyl acetals and acyl lactams are also preferred. Combinations of conventional bleach activators can also be used. Such bleach activators can be contained in the usual quantity range, preferably in amounts of 0.1 wt.-% to 10 wt.-%, in particular 2 wt.-% to 8 wt.-%, based on the total weight of the composition or agent.

In addition to or in place of the conventional bleach activators mentioned above, sulfonimines and/or bleach-enhancing transition metal salts or transition metal complexes may also be contained as so-called bleaching catalysts.

In preferred embodiments, the composition or agents comprise at least one enzyme or a mixture of enzymes.

Preferred enzymes provide cleaning performance and/or fabric care benefits. Examples of suitable enzymes include, but are not limited to, hemicellulases, peroxidases, amylases, proteases, cellulases, lipases, esterases, cutinases, pectinases, oxidases, pullulanases, or mixtures thereof. Suitable enzymes are enzymes derived from fungi or bacteria such as Bacillus subtilis, Bacillus lichenformis, Streptomyces griseus, Humicola lanuginosa, Humicola insolens, Pseudomonas pseudoalcaligenes or Pseudomonas cepacia. A typical combination is an enzyme cocktail that may comprise, for example, a protease, a cellulose, a lipase and/or an amylase. Enzymes used may be adsorbed on carriers and/or embedded in enveloping substances. When present in the composition or agent, the aforementioned enzymes may be present in amounts from about 0.00001 wt.-% to about 5 wt.-%, from about 0.0001 wt.-% to about 2 wt.-% or even from about 0.001 wt.-% to about 0.5 wt.-%, based on the total weight of the composition or the agent.

The composition or agent may preferably comprise enzyme stabilizers. Any conventional enzyme stabilizer may be used, for example by the presence of water-soluble sources of calcium and/or magnesium ions in the finished fabric and home care products that provide such ions to the enzymes. In case of aqueous compositions comprising protease, a reversible protease inhibitor, such as a boron compound including borate, or preferably 4-formyl phenylboronic acid, phenylboronic acid and derivatives thereof, or compounds such as calcium formate, sodium formate and 1,2-propane diol can be added to further improve stability.

The composition or agent may contain optical brighteners, for example, derivatives of diaminostilbenedisulfonic acid or alkali metal salts thereof. For example, salts of 4,4′-bis(2-anilino-4-morpholino-1,3,5-triazinyl-6-amino)stilbene-2,2′-disulfonic acid or similarly structured compounds, which carry a diethanolamino group, a methylamino group, an anilino group or a 2-methoxyethylamino group instead of the morpholino group are suitable.

In different embodiment, stilbene derivatives or biphenyl derivatives are preferably comprised, more preferably in amounts of 0.001 to 0.4 wt-%, based on the total weight of the composition or agent.

In some embodiments, the composition or agent may comprise a structurant, preferably from 0.01 wt.-% to 5 wt.-%, more preferably from 0.1 wt.-% to 2 wt.-%. The structurant is typically selected from the group consisting of diglycerides, triglycerides, ethylene glycol distearate, microcrystalline cellulose, cellulose-based materials, microfiber cellulose, hydrophobically modified alkali-swellable emulsions such as Polygel W30 (3VSigma), biopolymers, xanthan gum, gellan gum, hydrogenated castor oil, derivatives of hydrogenated castor oil such as non-ethoxylated derivatives thereof and mixtures thereof, in particular, those selected from the group of hydrogenated castor oil, derivatives of hydrogenated castor oil, microfibullar cellulose, hydroxyfunctional crystalline materials, long chain fatty alcohols, 12-hydroxystearic acids, clays and mixtures thereof.

Suitable foam inhibitors include, for example, organopolysiloxanes, and mixtures thereof with microfine, optionally silanated silica and paraffin waxes, and mixtures thereof with silanated silica or bis fatty acid-alkylenediamides. Mixtures of various foam inhibitors are also preferably used, for example those made of silicones, paraffins or waxes. The foam inhibitors, preferably foam inhibitors containing silicone and/or paraffin, are preferably bound to a granular, water-soluble or dispersible carrier substance. In particular, mixtures of paraffin waxes and bistearyl ethylenediamides are preferred. Preferably, foam inhibitors are present in the composition or the agent in amounts of 0.001 to 10 wt.-%, based on the total weight of the composition or agent.

Preferably, the composition comprises chelating agents and/or crystal growth inhibitor. Suitable molecules include copper, iron and/or manganese chelating agents and mixtures thereof. Further suitable molecules include aminocarboxylates, aminophosphonates, succinates, salts thereof, and mixtures thereof. Non-limiting examples of suitable chelants for use herein include ethylenediaminetetracetates, N-(hydroxyethyl)ethylenediaminetriacetates, nitrilotriacetates, ethylenediamine tetraproprionates, triethylenetetraaminehexacetates, diethylenetriamine-pentaacetates, ethanoldiglycines, ethylenediaminetetrakis (methylenephosphonates), diethylenetriamine penta(methylene phosphonic acid) (DTPMP), ethylenediamine disuccinate (EDDS), hydroxyethanedimethylenephosphonic acid (HEDP), methylglycinediacetic acid (MGDA), diethylenetriaminepentaacetic acid (DTPA), salts thereof, and mixtures thereof.

The compositions may further contain dye transfer inhibitors, preferably in amounts of 0.1 wt.-% to 2 wt.-%, in particular 0.1 wt.-% to 1 wt.-%, based on the total weight of the composition or agent, which are polymers of vinylpyrrolidone, vinylimidazole, vinylpyridine-N-oxide or copolymers thereof in a preferred form.

Suitable greying inhibitors are for example water-soluble colloids of an organic nature, for example starch, glue, gelatin, salts of ether carboxylic acids or ether sulphonic acids of starch or cellulose or salts of acidic sulphuric esters of cellulose or starch. Water-soluble polyamides containing acid groups are also suitable for this purpose. Furthermore, starch derivatives other than those mentioned above can be used, for example aldehyde starches. Cellulose ethers such as carboxymethylcellulose (sodium salt), methylcellulose, hydroxyalkylcellulose and mixed ethers, such as methylhydroxyethylcellulose, methylhydroxypropylcellulose, methylcarboxymethylcellulose, and mixtures thereof, for example in amounts of 0.1 to 5 wt.-%, based on the total weight of the composition or agent, can be preferably used.

For the adjustment of a desired pH value, which does not result automatically from the mixture of the other components, the compositions and agents may contain system-compatible and environmentally compatible acids, preferably citric acid, acetic acid, tartaric acid, malic acid, lactic acid, glycolic acid, succinic acid, glutaric acid and/or adipic acid, but also mineral acids, preferably sulfuric acid, or bases, preferably ammonium or alkali hydroxides. Such pH regulators are optionally contained in the composition or agent, preferably not more than 20 wt.-%, preferably from 0.2 wt.-% to 17 wt.-%.

In addition, the composition may comprise further discoloration inhibitors, antioxidants, metal sequestering agents, or other stabilizers to prolong the fragrance effect or to maintain the coloration of compositions or agents. However, the addition of further stabilizers is not preferred.

Furthermore, a laundry product is claimed, comprising a pro-fragrance compound or a composition.

In one preferred embodiment, the amount of the pro-fragrance compound is in the range of 0.001 to 5 wt.-%, more preferably in the range of 0.0015 to 3 wt.-%, most preferably in the range of 0.01 to 2 wt.-%, based on the total weight of the laundry product.

Preferably, the laundry product is a laundry detergent or a laundry after treatment agent, such as a softener.

Additionally, a home care product may include a pro-fragrance compound according to the invention, or a composition.

In one preferred embodiment, the amount of the pro-fragrance is in the range of 0.001 to 5 wt.-%, more preferably in the range of 0.0015 to 3 wt.-%, most preferably in the range of 0.01 to 2 wt.-%, based on the total weight of the home care product.

Preferably, the home care product is a dishwashing detergent, a cleaning agent or an air care product.

An insect repellant comprising a pro-fragrance compound or a composition is claimed as well.

In one preferred embodiment, the amount of the pro-fragrance compound is in the range of 0.001 to 99.9 wt.-%, more preferably in the range of 0.01 to 85 wt.-%, more preferably in the range of 0.1 to 70 wt.-%, most preferably in the range of 1 to 50 wt.-%, based on the total weight of the insect repellant.

The specified amount of the pro-fragrance compound relates to the amount of the pro-fragrance compound, which is added to the composition or agent, if not explicitly stated otherwise. Subsequent reduction of the pro-fragrance amount, for example based on degradation, precipitation and/or hydrolysis processes in the composition, has no influence on the feasibility of the non-limiting embodiments and will not limit the scope of the claims.

Every feature, which is described for the pro-fragrance compound, may also pertain to the composition and to the laundry product, home care product and/or insect repellant and vice versa.

In addition, a method for preparing a pro-fragrance compound is claimed, comprising or consisting of the following steps:

(i) reacting a compound of formula (XIII)

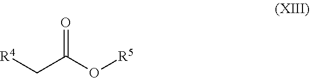

with a fragrance compound of formula (II),

wherein R⁴ is an electron-withdrawing group, as defined above, preferably an ester, a thioester, a semicarbamide, a hydrazone, an amide, a nitro group, a ketone, an aldehyde, a halogen atom, —CF₃, —C≡N, or a carboxylic acid, more preferably R⁴ is an ester, —CF₃, —F, —Cl, or —C≡N, most preferably R⁴ is —C≡N;

and wherein R⁵ is selected from H, or a linear or branched, saturated or unsaturated, substituted or unsubstituted hydrocarbon groups including up to 20 carbon atoms and optionally up to 6 heteroatoms,

preferably R⁵ is selected from linear or branched, substituted or unsubstituted alkyl, alkenyl or alkynyl groups including up to 20, preferably up to 12 carbon atoms, linear or branched, substituted or unsubstituted heteroalkyl, heteroalkenyl or heteroalkynyl groups including up to 20, preferably up to 12 carbon atoms, and 1 to 6, preferably 1 to 4 heteroatoms selected from O, S and N, substituted or unsubstituted cycloalkyl, cycloalkenyl or aryl including groups up to 20, preferably up to 12 carbon atoms, or substituted or unsubstituted heterocycloalkyl, heterocycloalkenyl or heteroaryl groups including up to 20, preferably up to 12 carbon atoms, and 1 to 6, preferably 1 to 4 heteroatoms selected from O, S and N, linear or branched, substituted or unsubstituted alkylcycloalkyl, alkenylcycloalkyl, alkylcycloalkenyl, alkenylcycloalkenyl, alkylaryl or alkenylaryl groups including up to 20, preferably up to 12 carbon atoms, or linear or branched, substituted or unsubstituted heteroalkylcycloalkyl, heteroalkenylcycloalkyl, heteroalkylcycloalkenyl, heteroalkenylcycloalkenyl, heteroalkylaryl or heteroalkenylaryl groups including up to 20, preferably up to 12 carbon atoms, and 1 to 6, preferably 1 to 4 heteroatoms selected from O, S and N, or linear or branched, substituted or unsubstituted heteroalkylheterocycloalkyl, heteroalkenylheterocycloalkyl, heteroalkylheterocycloalkenyl, heteroalkenylheterocycloalkenyl, heteroalkylheteroaryl or heteroalkenylheteroaryl groups including up to 20, preferably up to 12 carbon atoms, and 1 to 6, preferably 1 to 4 heteroatoms selected from O, S and N, more preferably R⁵ is selected from linear or branched, substituted or unsubstituted alkyl groups including up to 20, preferably up to 6 carbon atoms, more preferably R⁵ is ethyl or methyl, most preferably R⁵ is ethyl.

In case, one or more groups of R⁵ are substituted, the substituent can be chosen from every suitable substituent that is known to the person skilled and which have been defined above. Preferably R⁵ is unsubstituted.

Another aspect relates to the use of a pro-fragrance compound for increasing the longlastingness of a fragrance in compositions, preferably in laundry products, home care products or insect repellants, preferably for up to 48 hours, more preferably for up to 120 hours, in comparison to a corresponding free fragrance compound according to formula (II).

Another aspect is the use of a pro-fragrance compound for increasing the stability of a fragrance in compositions, preferably in laundry products, home care products or insect repellants, preferably for up to 48 hours, more preferably for up to 120 hours, in comparison to a corresponding free fragrance compound according to formula (II).

Since the ester-containing protection or anchor group of the pro-fragrance compound avoids or reduces the degradation, alteration or evaporation of the fragrance compound in compositions, the fragrance effect can be maintained over a prolonged period of time, in comparison to a free fragrance compound, which quickly evaporates or can be degraded or altered in the composition over time. Preferably, the pro-fragrance compound enables the release of the fragrance compound at a desired point of time, more preferably by hydrolysis.

Another aspect is the use of a pro-fragrance compound for improving the adhesion of a fragrance to textiles, in comparison to a corresponding free fragrance compound according to formula (II).

Preferably, the pro-fragrance compound can be used in a temperature range of −10° C. to 110° C., more preferably in a temperature range of 0° C. to 60° C., most preferably in a temperature range of 0.1° C. to 45° C.

### EXAMPLES

General Procedure for Preparing the Pro-Fragrance Compounds (Knoevenagel Condensation)

The fragrance aldehyde/ketone of interest (1 eq.), ethyl cyanoacetate (1.1 eq.-6 eq.) and ammonium acetate (0.5 eq.-3 eq.) in a mixture of acetic acid (1 eq.-3 eq.) and toluene (150 mL) were refluxed with azeotropic removal of H₂O in a Dean-Stark trap. The reaction mixture was cooled to room temperature (RT), washed with brine, dried over Na₂SO₄ and filtered. Subsequently, the solvent was evaporated. The pure products were isolated via flash column chromatography (hexane-ethyl acetate (EtOAc)-1% triethylamine) unless otherwise noted. All compounds were found to be cis/trans isomers at the newly formed double bound. Depicted structures however reflect only one isomer.

### Example 1: Ethyl-2-cyano-3-methyl-5-(2,6,6-trimethylcyclohex-1-en-1-yl)pent-2-enoate

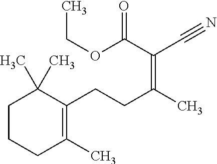 [SMILES: CCOC(=O)/C(C#N)=C(/C)CCC1=C(C)CCCC1(C)C]

Ethyl-2-cyano-3-methyl-5-(2,6,6-trimethylcyclohex-1-en-1-yl)pent-2-enoate was prepared by the aforementioned method using dihydro-beta-ionone and ethyl cyanoacetate (4 eq.). Until full conversion, the reaction was monitored by gas chromatography (GC) and after 17 h and 42 h, ammonium acetate (0.5 eq.) and acetic acid (1 eq.) were added and the mixture was allowed to react further.

Purified using gradient: 0%→5% EtOAc in hexane+1% Et₃N.

Pale yellow oil, 38% yield. cis/trans isomers. R_{f}=0.48 (10% EtOAc in hexane+1% Et₃N). ¹H-NMR (400 MHz, CDCl₃): δ=1.04 (s, 3H), 1.05 (s, 3H), 1.35 (dq, J=3.8, 7.1 Hz, 3H), 1.40-1.46 (m, 2H), 1.52-1.62 (m, 2H), 1.68 (s, 3H, isomer 1), 1.69 (s, 3H, isomer 2), 1.90-1.98 (m, 2H), 2.21-2.24 (m, 2H), 2.34 (s, 3H, isomer 1), 2.43 (s, 3H, isomer 2), 2.60-2.67 (m, 1H), 2.83-2.90 (m, 1H), 4.27 (q, J=7.1 Hz, 2H) ppm. ¹³C-NMR (CDCl₃, 125.8 MHz): 2 isomers: 6=14.1 (q), 19.3 (t), 19.8 (q), 21.0 (q), 25.2 (q), 26.7 (t), 28.5 (q), 32.9 (t), 35.1 (s), 35.9 (t), 39.8 (t), 41.2 (t), 104.2/104.7 (s), 115.7/115.9 (s), 129.0/129.3 (s), 135.0/135.6 (s), 161.4/162.1 (s), 176.6/177.2 (s). GC-MS: Optima 5 Accent 815, He, Split 1:10; 50° C.-12° C./min-325° C.; t_{R}=15.9 min, 16.0 min. MS (EI, 70 eV) m/z (%) at 15.9 min and at 16.0 min similar: 289 (10) [M]⁺, 137 (100), 121 (10), 107 (10), 95 (71), 81 (47), 67 (17), 41 (20).

### Example 2: Ethyl-cyano[3-(2-methoxy-2-oxoethyl)-2-pentylcyclopentylidene]ethanoate

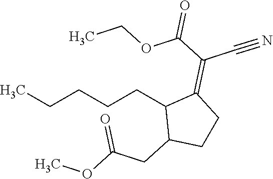 [SMILES: CCCCCC1/C(=C(/C#N)C(=O)OCC)CCC1CC(=O)OC]

Ethyl-cyano[3-(2-methoxy-2-oxoethyl)-2-pentylcyclopentylidene]ethanoate was prepared by the aforementioned method using hedione and ethyl cyanoacetate (4 eq.). Until full conversion, the reaction was monitored by GC and after 17 h and ammonium acetate (0.5 eq.) and acetic acid (1 eq.) were added and the mixture was allowed to react further.

Purified using gradient: 0%→5% EtOAc in hexane+1% Et₃N.

Pale yellow oil, 34% yield. cis/trans isomers. R_{f}=0.31 (10% EtOAc in hexane+1% Et₃N). ¹H NMR (400 MHz, CDCl₃): δ=0.85-0.93 (m, 3H), 1.20-1.68 (m, 8H), 1.34 (t, J=7.1 Hz, 3H, isomer 1), 1.35 (t, J=7.1 Hz, 3H, isomer 2), 2.05-2.30 (m, 3H), 2.47-2.63 (m, 1H), 2.76-2.82 (m, 1H), 2.83-2.89 (m, 1H), 2.94-3.17 (m, 2H), 3.68 (s, 3H, isomer 1), 3.69 (s, 3H, isomer 2), 4.27 (q, J=7.1 Hz, 2H). GC: Optima 5 Accent 815; 50° C.-12° C./min-325° C.; t_{R}=16.7 min, 16.9 min.

### Example 3: Ethyl-cyano(2-[3-butyl{2-methyl-(4 cyclohex-4-yliden)}]cyclopentylidene)ethanoate

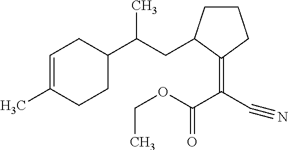 [SMILES: CCOC(=O)/C(C#N)=C1/CCCC1CC(C)C1CC=C(C)CC1]

Ethyl-cyano(2-[3-butyl{2-methyl-(4 cyclohex-4-yliden)}] cyclopentylidene)ethanoate was prepared by the aforementioned method using nectaryl and ethyl cyanoacetate (6 eq.). Until full conversion, the reaction was monitored by GC and after 17 h and 40 h, ammonium acetate (0.5 eq.) and acetic acid (1 eq.) were added and the mixture was allowed to react further.

Purified using gradient: 0%→5% EtOAc in hexane+1% Et₃N.

Pale yellow oil, 40% yield. cis/trans isomers. R_{f}=0.45 (10% EtOAc in hexane+1% Et₃N). ¹H-NMR (400 MHz, CDCl₃): δ=0.82-0.90 (m, 3H), 0.96-1.05 (m, 3H), 1.15-1.50 (m, 6H), 1.34 (t, J=7.1 Hz, 3H), 1.55-2.00 (m, 6H), 2.52 (q, J=7.1 Hz, 1H, isomer 1), 2.68-3.17 (m, 3H), 3.67 (m, 1H, isomer 1), 4.22-4.32 (m, 2H), 5.36 (s, 1H, isomer 2), 5.38 (s, 1H, isomer 1). GC-MS: Optima 5 Accent 815, 50° C.-12° C./min-320° C.; t_{R}=18.2 min, 18.5 min. MS (EI, 70 eV) m/z (%) at 18.2 min and at 18.5 min similar: 315 (27) [M]⁺, 242 (11), 192 (14), 121 (97), 95 (100), 81 (51), 67 (56), 55 (36), 41 (27).

### Example 4: Ethyl-2-cyano-5-(4-methoxyphenyl)-3-methylpent-2-enoate

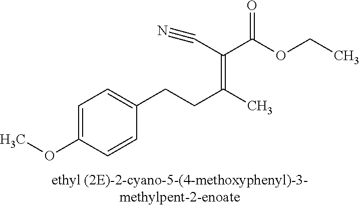 [SMILES: CCOC(=O)/C(C#N)=C(\C)CCc1ccc(OC)cc1]

Ethyl-2-cyano-5-(4-methoxyphenyl)-3-methylpent-2-enoate was prepared by the aforementioned method using frambinone methyl ether and ethyl cyanoacetate (1 eq.). Until full conversion, the reaction was monitored by GC and after 18 h and 66 h, ammonium acetate (0.5 eq.) and acetic acid (1 eq.) were added and the mixture was allowed to react further.

Purified using gradient: 0%→5% EtOAc in hexane+1% Et₃N

Brown oil, 72% yield. cis/trans isomers. R_{f}=0.33 (10% EtOAc in hexane+1% Et₃N). ¹H-NMR (400 MHz, CDCl₃): δ=1.34 (t, J=7.2 Hz, 3H), 2.25 (s, 3H, isomer 1), 2.38 (s, 3H, isomer 2), 2.70-2.79 (m, 1H), 2.83 (s, 2H), 3.01-3.08 (m, 1H), 3.79 (s, 3H), 4.26 (q, J=7.2 Hz, 2H), 6.80-6.87 (m, 2H), 7.10-7.18 (m, 2H). GC: Agilent HP 5, 50° C.-20° C./min-300° C.; t_{R}=11.0 min.

### Example 5: Ethyl-cyano(2-heptylcyclopentylidene)ethanoate

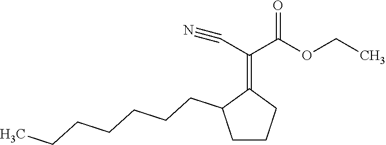 [SMILES: CCCCCCCC1CCC/C1=C(/C#N)C(=O)OCC]

Ethyl-cyano(2-heptylcyclopentylidene)ethanoate was prepared by the aforementioned method using fleuramone and ethyl cyanoacetate (1.1 eq.). Until full conversion, the reaction was monitored by GC and after 66 h, 72 h and 90 h, ammonium acetate (0.5 eq.) and acetic acid (1 eq.) were added and the mixture was allowed to react further.

Purified using gradient: 0%→5% EtOAc in hexane+1% Et₃N

Pale yellow oil, 37% yield. cis/trans isomers. R_{f}=0.5 (10% EtOAc in hexane+1% Et₃N). ¹H-NMR (400 MHz, CDCl₃): δ=0.87 (t, J=7.1 Hz, 3H), 1.19-1.48 (m, 12H), 1.34 (t, J=7.1 Hz, 3H), 1.58-1.67 (m, 1H, isomer 1), 1.70-1.90 (m, 4H), 2.68-3.10 (m, 3H), 3.53-3.60 (m, 1H, isomer 1), 4.26 (q, J=7.1 Hz, 2H, isomer 2), 4.28 (q, J=7.1 Hz, 2H, isomer 1). GC: Agilent HP 5, 50° C.-20° C./min-300° C.; t_{R}=10.6 min, 10.7 min.

### Example 6: Ethyl-2-cyano-3-methyl-5-phenylpent-2-enoate

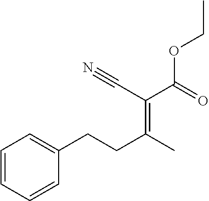 [SMILES: CCOC(=O)/C(C#N)=C(\C)CCc1ccccc1]

Ethyl-2-cyano-3-methyl-5-phenylpent-2-enoate was prepared by the aforementioned method using benzylacetone and ethyl cyanoacetate (1.1 eq.). Until full conversion, the reaction was monitored by GC. The reaction was stopped after 7 hours.

Purified using gradient: 0%→10% EtOAc in hexane+1% Et₃N.

Pale yellow oil, 71% yield, cis/trans isomers. R_{f}=0.4 (10% EtOAc in hexane+1% Et₃N). ¹H-NMR (400 MHz, CDCl₃) isomer 1: δ=1.18 (t, J=7.1 Hz, 3H), 2.11 (s, 3H, isomer 1), 2.19 (s, 3H, isomer 2), 2.89-2.91 (m, 1H), 2.92 (s, 2H), 3.02-3.06 (m, 1H), 4.12 (t, J=7.1 Hz, 2H), 7.08-7.17 (m, 5H) ppm. ¹³C-NMR (100.6 MHz, CDCl₃): δ=14.0 (q), 21.3 (q, isomer 1 or 2), 25.6 (q, isomer 1 or 2), 33.9 (t, isomer 1 or 2), 34.3 (isomer 1 or 2), 37.6 (t, isomer 1 or 2), 42.5 (t, isomer 1 or 2), 61.7 (t), 105.4 (s), 115.4 (s, isomer 1 or 2), 115.7 (isomer 1 or 2), 116.1 (d), 128.2 (d, 2C), 128.6 (d, 2C), 139.5 (s, isomer 1 or 2), 140.2 (s, isomer 1 or 2), 161.5 (s, isomer 1 or 2), 161.9 (s, isomer 1 or 2), 176.3 (s). GC: Agilent HP 5, 50° C.-20° C./min-300° C.; t_{R}=9.9 min.

### Example 7: Scent Tests of Knoevenagel Reaction Products

Method: Values given below are the average rating of 2 testers. Samples are prepared as 200 mM solutions in either ethanol (EtOH) or diethyl ether (Et₂O). The solution is absorbed onto scent test strips. Once the strips are soaked, the strips are removed and allowed to dry for 10 min. Subsequently, the scent impression was examined by trained test persons at the following intermittent periods.

TABLE 1
Overview of the examination results (scent impression) at different
time points based on strips with pro-fragrance substances and free
fragrance substances dissolved in EtOH or Et₂O.

| Solvent | Compound | 15 min | 1 h | 24 h | 48 h | 120 h |
|---|---|---|---|---|---|---|
| EtOH | Dihydro-ionone-CNEE- | 0.75 | 0.5 | 1 | 0.33 | 0.5 |
| | precursor | | | | | |
| | Dihydro-ionone | 5.5 | 4.5 | 0 | 0 | 0 |
| EtOH | Benzyl acetone-CNEE- | 4 | 4 | 4 | 4 | 3 |
| | precursor | | | | | |
| | Benzyl acetone | 5.5 | 4.5 | 1.5 | 0.33 | 1 |
| Et₂O | Dihydro-ionone-CNEE- | 1.5 | 1.25 | 1.25 | 0.33 | 0.25 |
| | precursor | | | | | |
| | Dihydro-ionone | 5.5 | 5 | 0.5 | 0 | 0 |
| Et₂O | Benzyl acetone-CNEE- | 4.25 | 4 | 4.5 | 4 | 3.5 |
| | precursor | | | | | |
| | Benzyl acetone | 6 | 5.5 | 1.5 | 0.66 | 1 |

### Example 8: Scent Test of Knoevenagel Reaction Products after Activation with pH 3 Buffer Solution

Method: Values given below are the average rating of 2 testers. Samples are prepared as 200 mM solutions in either EtOH or Et₂O. Solution is absorbed onto scent test strips. Once the strips are soaked, the strips are removed and allowed to dry for 10 min. Then, strips are sprayed with pH 3.0 buffer and the scent was examined by trained test persons at the following intermittent periods:

TABLE 2
Overview of the examination results (scent impression) at
different time points based on strips with pro-fragrance
substances and free fragrance substances dissolved in EtOH or
Et₂O und subsequent activation with pH 3 buffer solution.

| Solvent | Compound | 15 min | 1 h | 24 h | 48 h | 120 h |
|---|---|---|---|---|---|---|
| EtOH | Benzyl acetone-CNEE- | — | — | — | 4.5 | 2 |
| | precursor | | | | | |
| | Benzyl acetone | — | — | — | 4 | 0.5 |
| Et₂O | Dihydro-ionone-CNEE- | — | — | — | 1.5 | 0 |
| | precursor | | | | | |
| | Dihydro-ionone | — | — | — | 0 | 0 |
| Et₂O | Benzyl acetone-CNEE- | — | — | — | 3.5 | 2.5 |
| | precursor | | | | | |
| | Benzyl acetone | — | — | — | 2 | 0 |

Scale: 0-10, 0 relates to a non-noticeable scent impression and 10 is a strong scent impression.

1. A pro-fragrance compound of formula (I) 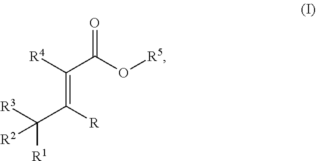
or a cis/trans isomer thereof,
wherein:
R, R¹, R², R³, and R⁵ are, independently of each other, selected from H, or linear or branched, saturated or unsaturated, substituted or unsubstituted hydrocarbon groups including up to 20 carbon atoms and optionally up to 6 heteroatoms;
or
R and one of the groups of R¹, R² and R³ form a substituted or unsubstituted hydrocarbon ring selected from substituted or unsubstituted cycloalkyl, cycloalkenyl or aryl groups including up to 12, carbon atoms, or substituted or unsubstituted heterocycloalkyl, heterocycloalkenyl or heteroaryl groups including up to 12 carbon atoms, and 1 to 6 heteroatoms selected from O, S, and N, or linear or branched, substituted or unsubstituted alkylcycloalkyl, alkenylcycloalkyl, alkylcycloalkenyl, alkenylcycloalkenyl, alkylaryl or alkenylaryl groups including up to 20 carbon atoms, or linear or branched, substituted or unsubstituted heteroalkylcycloalkyl, heteroalkenylcycloalkyl, heteroalkylcycloalkenyl, heteroalkenylcycloalkenyl heteroalkylaryl or heteroalkenylaryl groups including up to 20 carbon atoms, and 1 to 6 heteroatoms selected from O, S, and N, or linear or branched, substituted or unsubstituted heteroalkylheterocycloalkyl, heteroalkenylheterocycloalkyl, heteroalkylheterocycloalkenyl, heteroalkenylheterocycloalkenyl, heteroalkylheteroaryl or heteroalkenylheteroaryl groups including up to 20 carbon atoms, and 1 to 6 heteroatoms selected from O, S, and N;
wherein R⁴ is an electron-withdrawing group;
wherein at least one of R, R¹, R², and R³ is not hydrogen and that R, R¹, R², and R³ together with the carbon atoms to which they are attached are derived from a fragrance ketone or aldehyde of the formula (II) 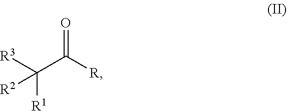
wherein R, R¹, R², and R³ are as defined above.

2. The pro-fragrance compound according to claim 1,
wherein R is linear or branched, substituted or unsubstituted alkyl including up to 20;
or
R and one of the groups of R¹, R² and R³ form a substituted or unsubstituted hydrocarbon ring selected from substituted or unsubstituted cycloalkyl, cycloalkenyl, or aryl groups including up to 6 carbon atoms, or linear or branched, substituted or unsubstituted alkylcycloalkyl, alkenylcycloalkyl, alkylcycloalkenyl, alkenylcycloalkenyl, alkylaryl or alkenylaryl groups including up to 20 carbon atoms, or linear or branched, substituted or unsubstituted heteroalkylcycloalkyl, heteroalkenylcycloalkyl, heteroalkylcycloalkenyl, heteroalkenylcycloalkenyl, heteroalkylaryl or heteroalkenylaryl groups including up to 20 carbon atoms, and 1 to 6 heteroatoms selected from O, S, and N;
wherein R⁵ is a linear or branched, substituted or unsubstituted alkyl including up to 20;
wherein at least one of R, R¹, R², and R³ is not hydrogen and that R, R¹, R², and R³ together with the carbon atoms to which they are attached are derived from a fragrance ketone or aldehyde of the formula (II).

3. The pro-fragrance compound according to claim 1, wherein the compound is a compound of formula (III): 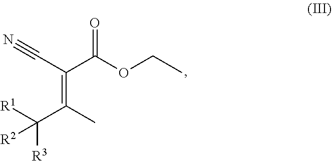
or a cis/trans isomer thereof,
wherein R¹, R², and R³ are, independently of each other, selected from hydrogen, linear or branched, substituted or unsubstituted alkyl, alkenyl, or alkynyl groups including up to 20 carbon atoms, linear or branched, substituted or unsubstituted heteroalkyl, heteroalkenyl, or heteroalkynyl groups including up to 20 carbon atoms, substituted or unsubstituted cycloalkyl, cycloalkenyl, or aryl groups including up to 20 carbon atoms, linear or branched, substituted or unsubstituted alkylcycloalkyl, alkenylcycloalkyl, alkylcycloalkenyl, alkenylcycloalkenyl, alkylaryl or alkenylaryl groups including up to 20 carbon atoms, or linear or branched, substituted or unsubstituted heteroalkylcycloalkyl, heteroalkenylcycloalkyl, heteroalkylcycloalkenyl, heteroalkenylcycloalkenyl, heteroalkylaryl or heteroalkenylaryl groups including up to 20 carbon atoms, and 1 to 6 heteroatoms selected from O, S, and N,
wherein at least one of R¹, R², and R³ is not hydrogen and that R¹, R², and R³ together with the carbon atom to which they are attached are derived from the corresponding group (R³R²R¹)C— in a fragrance ketone of the formula (IV)

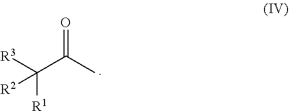

4. The pro-fragrance compound according to claim 1 of formula (V): 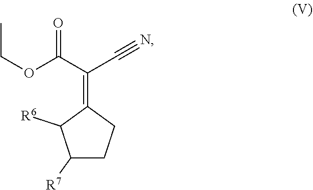
or a cis/trans isomer thereof,
wherein R⁶ and R⁷ are, independently of each other, selected from hydrogen, linear or branched, substituted or unsubstituted alkyl, alkenyl, alkylcycloalkyl, alkylcycloalkenyl, alkylaryl, alkenylcycloalkyl, alkenylcycloalkenyl or alkenylaryl groups including up to 15 carbon atoms or linear or branched, substituted or unsubstituted heteroalkyl, heteroalkenyl, heteroalkylcycloalkyl, heteroalkylcycloalkenyl, heteroalkylaryl, heteroalkenylcycloalkyl, heteroalkenylcycloalkenyl or heteroalkenylaryl groups including up to 15 carbon atoms, and 1 to 6 selected from O, S, and N;
wherein at least one of R⁶ and R⁷ is not hydrogen and that R⁶ and R⁷ together with the cyclopentyl group to which they are attached are derived from a fragrance ketone of the formula (VI)

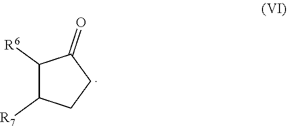

5. The pro-fragrance compound according to claim 1, wherein the pro-fragrance compound is configured to release a fragrance compound according to any of formulae (II), (IV), or (VI).

6. The pro-fragrance compound according to claim 1, wherein the fragrance compound of formula (II), (IV) or (VI) is selected from methyl-beta-naphthyl ketone, muskindanone (1,2,3,5,6,7-hexahydro-1,1,2,3,3-pentamethyl-4H-inden-4-one), tonalid (6-acetyl-1,1,2,4,4,7-hexamethyltetralin), alpha-damascone, beta-damascone, delta-damascone, iso-damascone, damascenone, hedione (methyl dihydrojasmonate), menthone (2-isopropyl-5-methylcyclohexanone), carvone (methyl-5-(prop-1-en-2-yl)cyclohex-2-enone), camphor, koavon (3,4,5,6,6-pentamethylhept-3-en-2-one), fenchone, alpha-ionone, beta-ionone, gamma-methyl-ionone, dihydro-beta-ionone, fleuramone (2-heptylcyclopentanone), frambinone (4-(4-hydroxy-phenyl-butan-2-one), frambinone methyl ether, dihydrojasmone, cis-jasmone, 1-(1,2,3,4,5,6,7,8-octahydro-2,3,8,8-tetramethyl-2-naphthalenyl)-ethan-1-one and isomers thereof, methylcedrenylketone, acetophenone, methylacetophenone, para-methoxyacetophenone, methyl-beta-naphtylketone, benzyl acetone, benzophenone, para-hydroxyphenylbutanone, celery-ketone (3-methyl-5-propylcyclohex-2-en-1-one), 6-isopropyldecahydro-2-naphtone, dimethyloctenone, frescomenthe (2-butane-2-yl-cyclohexane-1-one), 4-(1-ethoxyvinyl)-3,3,5,5-tetramethylcyclohexanone, methylheptenone, 2-(2-(4-methyl-3-cyclohexene-1-yl)propyl)cyclopentanone, 1-(p-menthene-6(2)yl)-1-propanone, 4-(4-hydroxy-3-methoxyphenyl)-2-butanone, nectaryl (2-(2-(4-methyl-3-cyclohexen-1-yl)propyl)cyclopentanone), 2-acetyl-3,3-dimethylnorbornane, 6,7-dihydro-1,1,2,3,3-pentamethyl-4-(5H)-indanone, 4-damascol, dulcinyl (4-(1,3-benzodioxol-5-yl)butane-2-one), hexalone (1-(2,6,6-trimethyl-2-cyclohexene-1-yl)-1,6-heptadiene-3-one), isocyclemone E 1-(2,3,8,8-tetramethyl-1,2,3,4,5,6,7,8-octahydro-2-naphthalenyl)ethanone, methylnonylketone, methylcyclocitrone, methyllavendelketone, orivone (4-tert-amylcyclohexanone), 4-tert-butyl cyclohexanone, delphone (2-pentyl cyclopentanone), muscone (CAS 541-91-3), neobutenone (1-(5,5-dimethyl-1-cyclo-hexenyl)pent-4-en-1-one), plicatone (CAS 41724-19-0), veloutone (2,2,5-trimethyl-5-pentylcyclopentane-1-one), 2,4,4,7-tetramethyl-oct-6-en-3-one, and tetrameran (6,10-dimethylundecene-2-one).

7. The pro-fragrance compound according to claim 1, having one of the following formulae (VII) to (XII) or a cis/trans isomer thereof,

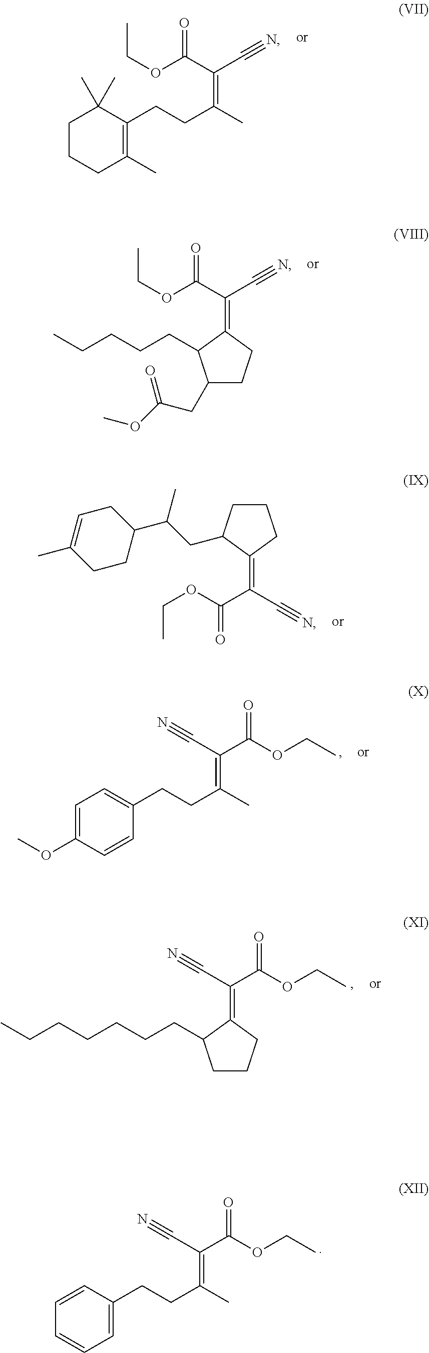

8. A composition comprising a pro-fragrance compound according to claim 1.

9. The composition according to claim 8, wherein the composition is a liquid state or a solid state at 25° C.

10. The composition according to claim 8, wherein the composition comprises at least one further component selected from one or more of: anionic surfactants, cationic surfactants, amphoteric surfactants, nonionic surfactants, acidifiers, alkalizing agents, anti-crease compounds, antibacterial/antimicrobial substances, antioxidants, antideposition agents, antistatic agents, bitter substances, bleaching agents, bleach activators, bleach stabilizers, bleach catalysts, builder substances, corrosion inhibitors, ironing aids, cobuilders, further fragrances or pro-fragrances, shrinkage inhibitors, electrolytes, emulsifiers, enzymes, enzyme stabilizers, protease inhibitors, colorants, dyes, dye transfer inhibitors, fluorescent agents, fungicides, germicides, metal sequestering agents, odor-complexing substances, auxiliary agents, hydrotropics, rinse aids, complexing agents, preservatives, optical brighteners, perfume carriers, pearl-luster agents, pH control agents, phobing and impregnating agents, polymers, swelling and sliding fasteners, foam inhibitors, layered silicates, dirt-repellent substances, organic solvents, silicone oils, soil-release active substances, UV-protective substances, viscosity regulators, thickeners, discoloration inhibitors, greying inhibitors, structurants, vitamins, fabric softeners, water; or combinations thereof.

11. The composition of claim 8, wherein the composition is a laundry composition, a home care composition, an insect repellant composition, or combinations thereof.

12. The composition of claim 11, wherein the amount of the pro-fragrance compound ranges from 0.001 to 5 wt.-% based on the total weight of the composition.

13. The composition of claim 11, wherein the amount of the pro-fragrance compound ranges from 0.001 to 99.9 wt.-% based on the total weight of the composition.

14. A method for preparing a pro-fragrance compound according to claim 1, wherein the method comprise:
(i) reacting a compound of formula (XIII) 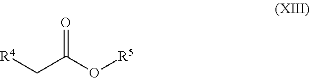
with a fragrance compound of formula (II) as defined in claim 1: wherein R⁴ and R⁵ are the same as defined in claim 1.

15. (canceled)

16. The composition of claim 8, wherein R, R¹, R², R³ and R⁵ are selected from linear or branched, substituted or unsubstituted alkyl, alkenyl or alkynyl groups including up to 20 carbon atoms, linear or branched, substituted or unsubstituted heteroalkyl, heteroalkenyl or heteroalkynyl groups including up to 20 carbon atoms, and 1 to 6 heteroatoms selected from O, S, and N, substituted or unsubstituted cycloalkyl, cycloalkenyl or aryl groups including up to 20 carbon atoms, or substituted or unsubstituted heterocycloalkyl, heterocycloalkenyl or heteroaryl groups including up to 20 carbon atoms, and 1 to 6 heteroatoms selected from O, S, and N, or linear or branched, substituted or unsubstituted alkylcycloalkyl, alkenylcycloalkyl, alkylcycloalkenyl, alkenylcycloalkenyl, alkylaryl or alkenylaryl groups including up to 20 carbon atoms, or linear or branched, substituted or unsubstituted heteroalkylcycloalkyl, heteroalkenylcycloalkyl, heteroalkylcycloalkenyl, heteroalkenylcycloalkenyl, heteroalkylaryl or heteroalkenylaryl groups including up to 20 carbon atoms, and 1 to 6 heteroatoms selected from O, S, and N, or linear or branched, substituted or unsubstituted heteroalkylheterocycloalkyl, heteroalkenylheterocycloalkyl, heteroalkylheterocycloalkenyl, heteroalkenylheterocycloalkenyl, heteroalkylheteroaryl or heteroalkenylheteroaryl including groups up to 20 carbon atoms, and 1 to 6 heteroatoms selected from O, S, and N.

17. The composition of claim 8, wherein R is methyl or ethyl.

18. The composition of claim 17, wherein R is methyl.
